(19) United States
(12) Patent Application Publication
(10) Pub. No.: US 2026/0069669 A1
(43) Pub. Date: Mar. 12, 2026

(54) ORAL VACCINE FOR ENHANCING CANCER IMMUNOTHERAPY

(71) Applicant: National Tsing Hua University, Hsinchu City (TW)

(72) Inventors: Hsing-Wen Sung, Hsinchu City (TW); Po Kai Luo, Hsinchu City (TW); Wan An Chang, Hsinchu City (TW)

(73) Assignee: National Tsing Hua University, Hsinchu City (TW)

(21) Appl. No.: 18/889,413

(22) Filed: Sep. 19, 2024

(30) Foreign Application Priority Data
Sep. 10, 2024 (TW) 113134186

(51) Int. Cl. A61K 39/00; A61K 9/00; A61K 9/51; A61K 36/06; A61P 37/04

(52) U.S. Cl. CPC A61K 39/0011; A61K 9/0053; A61K 9/5123; A61K 36/06; A61P 37/04; A61K 2039/53; A61K 2039/542; A61K 2039/6087

(57) ABSTRACT

The disclosure provides an oral vaccine for enhancing cancer immunotherapy, which includes a complex of β-glucan and mRNA and lipid nanoparticles, wherein the mRNA includes a coding region of a tumor antigen, and the lipid nanoparticles encapsulate the complex of β-glucan and mRNA.

### CROSS-REFERENCE TO RELATED APPLICATION

This application claims the priority benefit of Taiwan application serial no. 113134186, filed on Sep. 10, 2024. The entirety of the above-mentioned patent application is hereby incorporated by reference herein and made a part of this specification.

### BACKGROUND

### Technical Field

The disclosure relates to an oral vaccine, and particularly relates to an oral vaccine for enhancing cancer immunotherapy.

### Description of Related Art

Cancer vaccines are a form of cancer immunotherapy that mainly utilize partial or complete tumor antigens as vaccine components to enable the patient's immune system to recognize and attack cancer cells. Common cancer vaccines include protein vaccines, DNA vaccines, and mRNA vaccines. Among them, mRNA vaccines have the advantages of safety and flexibility in responding to mutations.

Currently, mRNA vaccines are mainly delivered using lipid nanoparticles (LNP), and intravenous delivery of mRNA@LNP is currently a common treatment method. However, since mRNA@LNP easily adsorbs ApoE in plasma, this will lead to its excessive accumulation in the liver, resulting in a large number of mRNA expression in the liver. Such distribution characteristics limit its contact opportunities with immune cells, thus weakening specific cellular immune responses. As such, such a limitation contributes to the poor performance of intravenously injected mRNA@LNP in inhibiting tumor growth.

On the other hand, traditional intravenous (IV) and intramuscular (IM) injection methods, although common, have low patient compliance and require professional medical personnel to operate. In addition, as mentioned above, these injection methods lead to large expression of nucleic acids in the liver and are unable to efficiently reach the immune cells. Oral delivery has advantages in these aspects, but if nucleic acid vaccines are delivered through the oral route, they need to face challenges in the gastrointestinal tract environment and immune response.

Based on the above, a new vaccine delivery strategy is proposed that not only solves the limitations of intravenous and intramuscular injection, but also overcomes the obstacles of oral delivery, which is an important issue for the development of technology in this field.

### SUMMARY

The disclosure provides an oral vaccine, which may effectively solve the shortcomings of existing nucleic acid vaccine delivery methods, eliminate the limitations of intravenous and intramuscular injection, and overcome the obstacles of oral delivery.

The oral vaccine for enhancing cancer immunotherapy of the disclosure includes a complex of β-glucan and mRNA and lipid nanoparticles, wherein the mRNA includes a coding region of a tumor antigen, and the lipid nanoparticles encapsulate the complex of β-glucan and mRNA.

In an embodiment of the disclosure, β-glucan is derived from yeast.

In an embodiment of the disclosure, a molar ratio of glucose units of the β-glucan to nucleotide bases of the mRNA is 15:1 to 120:1.

In an embodiment of the disclosure, a material of the lipid nanoparticles includes heptadecan-9-yl 8-((2-hydroxyethyl)(6-oxo-6-(undecyloxy)hexyl)amino)octanoate, 1,2-distearoyl-sn-glycero-3-phosphocholine, cholesterol, and 1,2-dimyristoyl-rac-glycero-3-methoxypolyethylene glycol-2000.

In an embodiment of the disclosure, a particle size of the lipid nanoparticles is 50 nm to 200 nm.

Based on the above, the disclosure proposes an oral vaccine that uses the oral route to deliver mRNA, which may directly contact high-density immune cells in the gastrointestinal tract, thereby effectively promoting cellular immune responses. At the same time, the oral vaccine of the disclosure uses yeast-derived β-glucans as an adjuvant and protective factor to create a stable βGlus/mRNA complex and further encapsulate it in the lipid nanoparticles (LNP) to form a highly stable and immune-modulating βGlus/mRNA@LNP carrier. Through the adjuvant and protective effects of β-glucan, it may protect mRNA in the violent chemical environment of the gastrointestinal tract (GI), improve the stability and transfection efficiency of mRNA in the gastrointestinal tract environment, and strengthen its ability to stimulate the immune system, thus demonstrating high effectiveness in triggering a powerful cellular immune response and anti-tumor effects.

### BRIEF DESCRIPTION OF THE DRAWINGS

FIG. 1A and FIG. 1B show CD spectra of βGlus/mRNA in solution or within lipid nanoparticles using a CD spectrometer under different conditions.

FIG. 2 is a diagram of a morphological structure of LNP evaluated using transmission electron microscope (TEM).

FIG. 3 is a graph showing expression evaluation of CD40 markers.

FIG. 4 is a graph showing expression evaluation of CD11c+ cells and CD107α+ cells.

### DESCRIPTION OF THE EMBODIMENTS

In the specification, scopes represented by “a numerical value to another numerical value” are schematic representations in order to avoid listing all of the numerical values in the scopes in the specification. Therefore, the recitation of a specific numerical range covers any numerical value in the numerical range and a smaller numerical range defined by any numerical value in the numerical range, as is the case with any numerical value and a smaller numerical range thereof in the specification.

The disclosure provides an oral vaccine for enhancing cancer immunotherapy, which includes a complex of β-glucan and mRNA and lipid nanoparticles. In the embodiment, the mRNA includes a coding region of a tumor antigen, and the tumor-specific antigen is, for example, ovalbumin (OVA) mRNA (mOVA). The lipid nanoparticles encapsulate the complex of β-glucan and mRNA. The lipid nanoparticles mainly serve as delivery carriers, surrounding and protecting the complex of β-glucan and mRNA to promote stable delivery to target cells, including dendritic cells.

In the embodiment, β-glucan is derived from yeast and serves as an adjuvant and protective factor. Through the adjuvant and protective effects of β-glucan, it protects mRNA in the violent chemical environment of the gastrointestinal tract (GI), improves the stability and transfection efficiency of mRNA in the gastrointestinal tract environment, and strengthens its ability to stimulate the immune system. The molar ratio of the glucose units of the β-glucan to the nucleotide bases of the mRNA is 15:1 to 120:1, so that β-glucan may fully exert its protective effect, and the encapsulation efficiency (EE) of mRNA in the lipid nanoparticles is 80% or more. Hydrogen bonding interactions occur between the amine groups of mRNA and the hydroxyl groups of β-glucan, resulting in the formation of the complex of β-glucan and mRNA.

In the embodiment, the material of the lipid nanoparticles includes heptadecan-9-yl 8-((2-hydroxyethyl)(6-oxo-6-(undecyloxy)hexyl)amino)octanoate, 1,2-distearoyl-sn-glycero-3-phosphocholine, cholesterol, and 1,2-dimyristoyl-rac-glycero-3-methoxypolyethylene glycol-2000. Heptadecan-9-yl 8-((2-hydroxyethyl)(6-oxo-6-(undecyloxy)hexyl)amino)octanoate, 1,2-distearoyl-sn-glycero-3-phosphocholine, cholesterol, and 1,2-dimyristoyl-rac-glycero-3-4-methoxypolyethylene glycol-2000, is, for example, formulated with a molar ratio of 50:10:38.5:1.5. The particle size of the lipid nanoparticles is 50 nm to 200 nm. In the preparation of the lipid nanoparticles, a microfluidic formulation method was used. First, a microfluidic device was used to mix the ethanol phase and aqueous phase at a flow ratio of 1:3. The ethanol phase consists of a four-component lipid (heptadecan-9-yl 8-((2-hydroxyethyl)(6-oxo-6-(undecyloxy)hexyl)amino)octanoate, 1,2-distearoyl-sn-glycero-3-phosphocholine, cholesterol, and 1,2-dimyristoyl-rac-glycero-3-methoxypolyethylene glycol-2000), while the aqueous phase contains sodium acetate solution (pH 5.0). The lipid nanoparticles were diluted in DPBS without Mg²⁺ and Ca²⁺, and then concentrated using a Centrifugal Filter. Afterwards, the lipid nanoparticles were stored in DPBS at 4° C. for subsequent use.

In order to increase the solubility of β-glucan, hydrogen peroxide was used as an oxidizing agent to react with β-glucan in zymosan. First, a solution was prepared by dissolving 10 g of sodium hydroxide (NaOH) and 500 mg of zymosan in 75 mL of deionized (DI) water, and the mixture was stirred thoroughly with a magnetic stirrer until completely dissolved. Subsequently, 25 mL of 30% (w/w %) hydrogen peroxide solution was added, and the mixture was magnetically stirred in a 60° C. oil bath for 60 hours. After the reaction, the degraded zymosan solution was dialyzed against reverse osmosis (RO) water at 4° C. for 48 hours. After dialysis, the solution was centrifuged at 2000 rpm for 5 min and the supernatant was collected while avoiding sediment. Then, the collected supernatant was freeze-dried to obtain β-glucan in dry powder form, which was subsequently stored at 4° C. until use.

In preparing test lipid nanoparticles for mRNA delivery, a lipid mixture containing heptadecan-9-yl 8-((2-hydroxyethyl)(6-oxo-6-(undecyloxy)hexyl)amino)octanoate, 1,2-distearoyl-sn-glycero-3-phosphocholine, cholesterol, and 1,2-dimyristoyl-rac-glycero-3-methoxypolyethylene glycol-2000 was formulated in a molar ratio of 50:10:38.5:1.5. The lipid mixture was dissolved in 99% ethanol solution to form an organic phase. At the same time, the mRNA and β-glucan were dissolved in sodium acetate buffer (30 mM) to form an aqueous phase, and a microfluidic device was used to combine the two phases to form test lipid nanoparticles for mRNA delivery.

After the oral vaccine of the disclosure is taken, when it enters the gastrointestinal tract, it will come into contact with the intestinal tract, the largest immune organ of the human body, and reach the mesenteric lymph nodes. This avoids liver accumulation problems associated with intravenous administration and triggers a stronger immune response. In addition, it enhances the immune stimulating function of the dendritic cells in the gastrointestinal tract, guides more dendritic cells to migrate to the mesenteric lymph nodes, and further stimulates a stronger cellular immune response in the spleen. In more detail, the β-glucan adjuvant in the oral vaccine of the disclosure may enhance the innate immune response of the dendritic cells and may actively interact with T cells in the mesenteric lymph nodes to promote the activation thereof so as to enhance the effect of cancer immunotherapy. At the same time, the mRNA in the oral vaccine of the disclosure is translated and processed by the dendritic cells into tumor antigen fragments, which are presented on the surface of the dendritic cells. Subsequently, the dendritic cells carrying the tumor antigen fragments flow into the mesenteric lymph nodes and stimulate the T cells, triggering a specific immune response against the presented antigen.

When the complex of β-glucan and mRNA of the disclosure is exposed to an acidic environment with a pH of 2.0 (gastric acid effect), in response to an acidic pH environment, RNA will autonomously transform from a typical single-stranded helix structure to a double-stranded state. Such a transformation results in the dissociation of the complex of β-glucan and mRNA, thereby releasing free β-glucan and mRNA molecules. After returning to neutral conditions (small intestine), the previously separated β-glucan and mRNA components showed a tendency to approach each other within the lipid nanoparticles. That is to say, in the acidic environment (gastric acid effect), the complex of β-glucan and mRNA may spontaneously dissociate, thereby achieving successful transfection of mRNA. When returned to the neutral conditions (small intestine), β-glucan acts as a protective agent. Therefore, β-glucan may protect the mRNA molecules within the lipid nanoparticles from degradation during gastrointestinal transport, thereby promoting stable delivery of mRNA into immune cells and transfection.

The oral vaccine proposed by the disclosure for enhancing cancer immunotherapy will be described in detail below through experimental examples. However, the following experimental examples are not intended to limit the disclosure.

### EXPERIMENTAL EXAMPLE

### Circular Dichroism Spectrum (CD Spectrum) Analysis

CD spectra of βGlus/mRNA in solution or within lipid nanoparticles were analyzed using a CD spectrometer (MOS-500, BioLogic, France) under neutral (pH 7.0) and acidic (pH 2.0) conditions. All samples were prepared in sodium acetate or DPBS buffer at a concentration of 200 μg/mL mOVA. CD spectra were recorded at room temperature using a cuvette with an optical path length of 0.1 cm and a wavelength range of 230-300 nm with an interval of 0.5 nm. The recorded CD signal is expressed in terms of ellipticity (θ) in millidegrees [x].

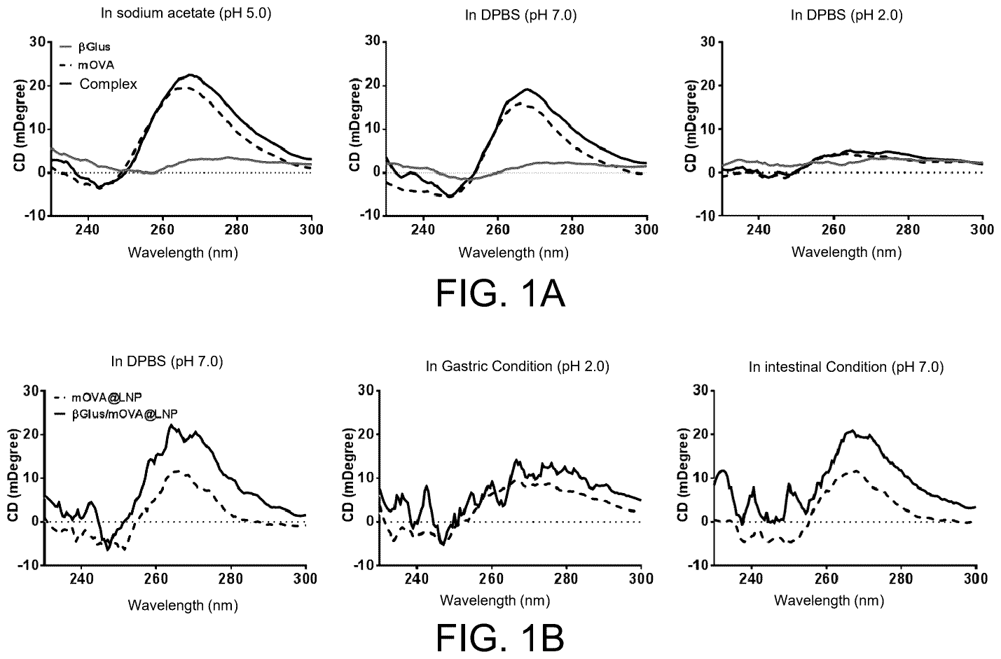

In the CD spectrum of mOVA in sodium acetate/DPBS aqueous solution, a characteristic band was detected in the 260-280 nm region, as shown in FIG. 1A. Under neutral pH conditions, RNA usually exists as a single-stranded molecule, while βGlus usually exists in a triple-helical conformation in aqueous solution. Notably, an increase in the intensity of the characteristic band was observed when the combined CD spectrum of βGlus and mOVA in aqueous solution at neutral pH was compared with their respective CD spectra. This enhanced intensity indicates that hydrogen bonding interactions begin to occur between the amine groups on mOVA and the hydroxyl groups on βGlus in aqueous solution, leading to the formation of βGlus/mOVA complexes. These complexes may then be encapsulated in a four-component lipid within the ethanol phase using a microfluidic device, ultimately producing βGlus/mRNA@LNPs as previously described.

However, when the βGlus/mOVA complexes were exposed to an acidic environment at pH 2.0 (in DPBS), the characteristic band of mOVA disappeared, and such a disappearance strongly suggested that their presence in aqueous solution was significantly reduced. In response to the acidic pH environment, RNA molecules undergo a transformation from a single-stranded helix to a double-stranded structure. The increase in hydrogen ions causes the protonation of their bases, promoting the formation of hydrogen bonds between them. Therefore, the RNA molecule transforms from a single-stranded state to a double-stranded state. Such a transformation results in the dissociation of the βGlus/mOVA complex, thereby releasing free βGlus and mOVA molecules. Notably, previous reports have demonstrated that in acidic water environments, free mRNA molecules tend to aggregate and subsequently precipitate.

The ability of βGlus/mRNA complexes within LNPs (βGlus/mOVA@LNPs) to withstand potential instability under different GI pH conditions was evaluated using CD spectroscopy. The circular dichroism spectra of βGlus/mOVA@LNPs were analyzed under neutral (pH 7.0) and acidic (pH 2.0) conditions. As a control, LNP lacking βGlus (mOVA@LNP) was also examined. As shown in FIG. 1B, the CD spectrum of mOVA@LNP under neutral pH conditions (in DPBS, storage conditions) is very similar to the CD spectrum of free mOVA, as shown in FIG. 1A. Notably, the CD band of βGlus/mOVA@LNPs increased in intensity at 260-280 nm compared with its mOVA@LNPs counterpart. Such an increase is similar to the CD spectral curves observed in aqueous solutions containing βGlus and mOVA compared to free mOVA. The observation indicates that βGlus and mOVA molecules in LNP are still complexed.

When the test LNPs were exposed to an acidic environment of pH 2.0 (simulating the pH of the gastric condition), they behaved differently from the βGlus/mOVA complex in aqueous solution (FIG. 1A). In the case of βGlus/mOVA@LNPs (FIG. 1B), the characteristic band at 260-280 nm still exists. However, the intensity of its CD spectrum is significantly reduced, reaching a level somewhat close to that of mOVA@LNPs but relatively high. The observations indicate that βGlus/mOVA complexes within LNPs do not aggregate or precipitate, possibly because they are individually confined to small compartments. On the contrary, mOVA appears to be partially dissociated from βGlus within the limited space of the LNP, which is different from the free mOVA observed in mOVA@LNP. When returned to neutral conditions (simulating the pH of the intestine), the circular dichroism spectral intensity of βGlus/mOVA@LNPs again showed an increasing trend, indicating the possibility of recombination of mOVA and βGlus complexes.

### Morphological Structure Evaluation

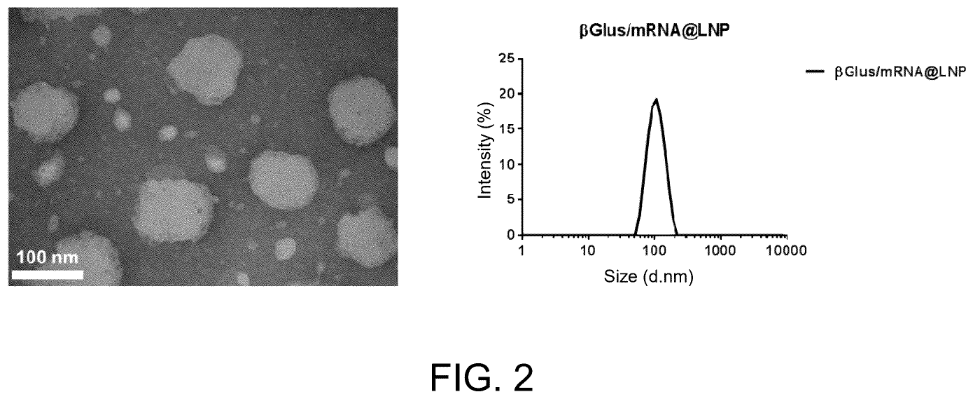

The morphological structure of the optimized LNPs was evaluated using transmission electron microscope (TEM). As shown in FIG. 2, both mOVA@LNPs and βGlus/mOVA@LNPs exhibit spherical morphology. The size and zeta potential values of these particles were determined by dynamic light scattering (DLS). As shown in FIG. 2, the size of βGlus/mOVA@LNPs is slightly larger (approximately 100 nm) and the zeta potential value is more negative (−4 mV) compared with mOVA@LNPs (80 nm and −2 mV).

### Evaluation of Expression of CD40 Markers

Next, the ability of mOVA@LNPs and βGlus/mOVA@LNPs to activate BMDCs was evaluated in vitro. The control group contained components of mOVA and βGlus, each present in equal amounts in the test particles. In addition, cells exposed only to the culture medium with DPBS replacing the test sample solution served as untreated controls. To simulate DC activation in vivo, test samples were first exposed to simulated gastric fluid (SGF) containing pepsin at pH 2.0. Subsequent exposure was to simulated intestinal fluid (SIF) containing lipase at pH 7.0. These exposures were maintained at 37° C. for specified time intervals (20 minutes for SGF, 40 minutes for SIF), consistent with biodistribution observations following oral administration. After exposure to SGF and SIF, TEM micrographs showed that the morphology of mOVA@LNPs and βGlus/mOVA@LNPs remained essentially unchanged compared to before treatment. However, their sizes are relatively larger as measured by DLS, with each size increasing by approximately 20 nm.

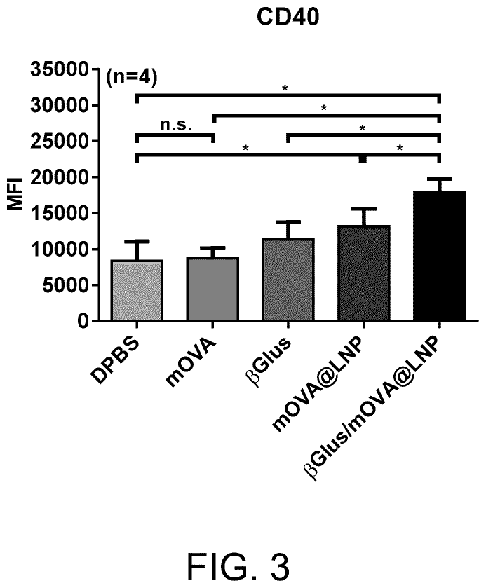

After activation, DCs upregulate the surface expression of CD40, which is critical for the immunostimulatory function of DCs. As shown in FIG. 3, exposure to naked mOVA did not significantly increase the expression of CD40 markers (P>0.05) on BMDCs compared to untreated cells. This lack of elevation may be attributed to the vulnerability of naked mOVA to degradation in the harsh simulated gastrointestinal tract environment, as well as challenges in cellular uptake due to its negatively charged nature and single-chain structure. In contrast, free βGlus effectively upregulated the expression of CD40 markers compared with untreated control group. βGlus is recognized by pattern recognition receptors on immune cells, including DCs, thereby triggering activation of the innate immune response. Such an adjuvant effect is expected to enhance vaccine efficacy, making βGlus a valuable candidate for vaccine development.

Compared with the untreated control group, both mOVA@LNPs and βGlus/mOVA@LNPs significantly increased the level of CD40 markers (P<0.05) on BMDCs, indicating that they were still efficiently taken up by cells despite the relative increase in particle size after exposure to SGF and SIF. Specifically, βGlus/mOVA@LNPs induced a significant increase in CD40 levels of 115% compared to the 55% increase observed with mOVA@LNPs. This suggests that, in addition to acting as an adjuvant (contributing to the 35% increase), βGlus may play a crucial protective role for complexed mOVA within LNPs when exposed to the harsh simulated gastrointestinal tract environment. As mentioned previously, such a protective effect may originate from the pH-responsive partial dissociation/reassociation behavior of βGlus/mOVA complexes within the limited space of LNP when exposed to different GI pH conditions. In addition, the unique pH-responsive behavior of βGlus/mOVA complex may help enhance the stability of its complexed mRNA.

Evaluation of Expression of CD11c⁺ Cells and CD107α⁺ Cells

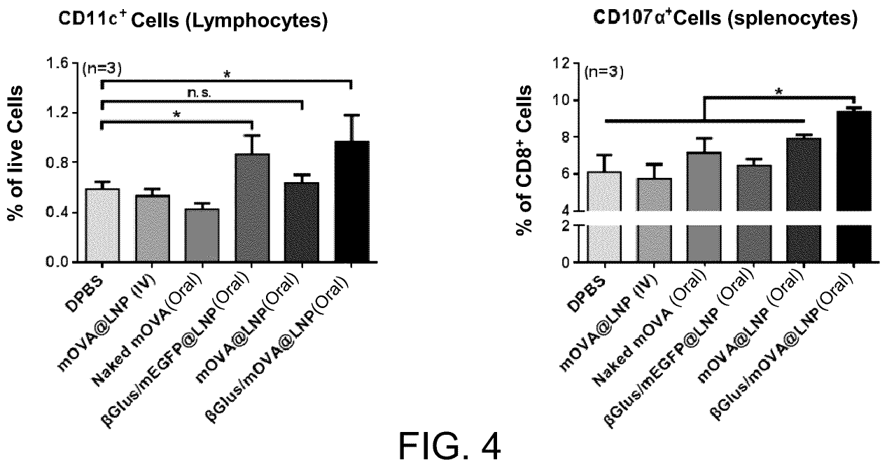

Test mice were immunized twice weekly with the oral formulation and once weekly with the intravenous formulation, according to the schedule, for a duration of two weeks. Seven days after the last oral boost, the animals were sacrificed and their MLNs were recovered for analysis by flow cytometry. FIG. 4 shows that an increased frequency of DC (CD11c⁺) was observed in the oral βGlus/mEGFP@LNPs or βGlus/mOVA@LNPs treatment group compared with the oral control group lacking βGlus (mOVA@LNPs). This suggests that incorporation of βGlus into LNPs may effectively activate immature DCs and initiate their migration to MLNs.

To evaluate the immune memory of cytotoxic T cells in test mice, splenocytes were collected, isolated, stimulated with OVA peptide, and then analyzed using flow cytometry. Such an analysis involves quantifying the immediate surface expression of CD107α⁺, a lysosome-associated membrane protein-1 marker expressed by stimulated CD8⁺ T cells. As shown in FIG. 4, after OVA peptide stimulation, the percentage of CD8⁺CD107α⁺ T cells in mice vaccinated with βGlus/mOVA@LNPs orally was significantly increased (P<0.05) compared with mice vaccinated with the control group. These findings jointly verify that the intensity of the in vivo adaptive immune response induced by oral administration of βGlus/mOVA@LNPs is significantly stronger than the intensity of the control group, indicating the potential use of the oral vaccine proposed in the disclosure in cancer immunotherapy.

In summary, the disclosure proposes an oral vaccine that uses the oral route to deliver mRNA, which may directly contact high-density immune cells in the gastrointestinal tract, thereby effectively promoting cellular immune responses. At the same time, the oral vaccine of the disclosure uses yeast-derived β-glucans as an adjuvant and protective factor to create a stable βGlus/mRNA complex and further encapsulate it in the lipid nanoparticles (LNP) to form a highly stable and immune-modulating βGlus/mRNA@LNP carrier. Through the adjuvant and protective effects of β-glucan, it may protect mRNA in the violent chemical environment of the gastrointestinal tract (GI), improve the stability and transfection efficiency of mRNA in the gastrointestinal tract environment, and strengthen its ability to stimulate the immune system, thus demonstrating high effectiveness in triggering a powerful cellular immune response and anti-tumor effects. In this way, the shortcomings of existing nucleic acid vaccine delivery methods may be effectively solved, the limitations of intravenous and intramuscular injection may be eliminated, and the obstacles of oral delivery may be overcome.

What is claimed is:

1. An oral vaccine for enhancing cancer immunotherapy, comprising:
a complex of β-glucan and mRNA, comprising a coding region of a tumor antigen; and
lipid nanoparticles, encapsulating the complex of β-glucan and mRNA.

2. The oral vaccine according to claim 1, wherein the β-glucan is derived from yeast.

3. The oral vaccine according to claim 1, wherein a molar ratio of glucose units of the β-glucan to nucleotide bases of the mRNA is 15:1 to 120:1.

4. The oral vaccine according to claim 1, wherein a material of the lipid nanoparticles comprises heptadecan-9-yl 8-((2-hydroxyethyl)(6-oxo-6-(undecyloxy)hexyl)amino)octanoate, 1,2-distearoyl-sn-glycero-3-phosphocholine, cholesterol, and 1,2-dimyristoyl-rac-glycero-3-methoxypolyethylene glycol-2000.

5. The oral vaccine according to claim 1, wherein a particle size of the lipid nanoparticles is 50 nm to 200 nm.
